(19) United States
(12) Patent Application Publication
(10) Pub. No.: US 2009/0088726 A1
(43) Pub. Date: Apr. 2, 2009

(54) SYMBIOTIC BIOLOGICAL SYSTEMS AS PLATFORMS FOR SENSING, PRODUCTION, AND INTERVENTION

(75) Inventors: Eric J. Horvitz, Kirkland, WA (US); Steven Bathiche, Kirkland, WA (US); David E. Heckerman, Bellevue, WA (US); Chris Demetrios Karkanias, Sammamish, WA (US)

Correspondence Address: AMIN, TUROCY & CALVIN, LLP, 127 Public Square, 57th Floor, Key Tower, CLEVELAND, OH, 44114, US

(73) Assignee: MICROSOFT CORPORATION, Redmond, WA (US)

(21) Appl. No.: 11/864,669

(22) Filed: Sep. 28, 2007

(51) Int. Cl. A61M 5/00; A61B 5/00

(52) U.S. Cl. 604/522; 600/300

(57) ABSTRACT

Provided are systems and/or methods that facilitate sensing, detecting, or treatment of a condition or need of a living body using a genetically engineered symbiotic agent.

### BACKGROUND

A gene is the basic physical and functional unit of heredity. Genes are specific sequences of bases (DNA) that encode instructions on how to make proteins. The body retains genes in the nucleus of every cell, and organizes them in the chromosomes that hold the DNA. In humans, genes vary in size from a few hundred DNA bases to more than tens of thousands of genes. When genes are altered so that the encoded proteins are unable to carry out their normal functions, genetic disorders can result.

Recombinant DNA technology has made the transfer of genes from one organism to another possible. The use of recombinant DNA technology in conjunction with humans and other mammals is referred to as gene therapy. Gene therapy is a technique for correcting defective genes responsible for disease development. Although researchers may use one of several approaches for correcting faulty genes, most commonly a normal gene is inserted into a nonspecific location within the genome to replace a nonfunctional or malfunctioning gene. That is, in gene therapy, a normally functioning gene is inserted into the genome to replace an abnormally functioning or disease-causing gene. A carrier molecule called a vector is used to deliver the therapeutic gene to the patient's target cells. The most common vector is a virus that is genetically altered to carry normal human DNA. Viruses are employed because they function by encapsulating and delivering their genes to human cells typically in a pathogenic manner. Scientists take advantage of this functionality and manipulate the virus genome to remove disease-causing genes and insert therapeutic genes.

For example, target cells usually from a tissue affected by the illness such as the patient's liver or lung cells are infected with a viral vector containing a therapeutic gene of interest. The vector then unloads its genetic material containing the therapeutic human gene into the target cell. The generation of a functional protein product from the therapeutic gene restores the target cell to a normal state.

One problem is that gene therapy is irreversible. In other words, once a new gene is introduced into a patient's genome, it persists indefinitely and cannot be removed. Other problems with gene therapy include toxicity concerns as well as vector-induced inflammation and immune responses.

Also in the context of addressing illnesses, the growing number of resistances of microorganisms against known antibiotics is a problem. Currently, much effort is underway to further develop active substances to ensure sufficient protection for humans and animals from infection. In addition, the use of conventional antibiotics in animals is extremely disputed because a possibility exists that this results in the final member of the food chain, namely humans, to be excessively subjected to antibiotics or antibiotic metabolites, which may exacerbate additional antibiotic resistances.

### SUMMARY

The following presents a simplified summary of the innovation in order to provide a basic understanding of some aspects described herein. This summary is not an extensive overview of the claimed subject matter. It is intended to neither identify key or critical elements of the claimed subject matter nor delineate the scope of the subject innovation. Its sole purpose is to present some concepts of the claimed subject matter in a simplified form as a prelude to the more detailed description that is presented later.

The subject innovation relates to systems and/or methods that facilitate monitoring, sensing, signaling, and/or treatment of conditions in a living body. A symbiotic agent can monitor, sense, signal, and/or treat various conditions and further or alternatively provide information indicative of at least one condition of the living body. The symbiotic agent can generate information for communication with an external system, which can use the information for diagnostic, experimental, research, and/or preventative medicine. Unlike the genetic material in gene therapy, however, a genetically engineered symbiotic agent can be removed from a living body or controllably deactivated.

In accordance with one aspect of the claimed subject matter, a system that facilitates sensing, detecting, or treatment of a condition of a living body is provided. The system employs a symbiotic agent, an interface component that facilitates receipt of or delivery of data associated with the condition of the living body, and an operation component that analyzes received data associated with the condition of the living body or provides data for delivery to the symbiotic agent.

In accordance with another aspect of the claimed subject matter, methods that facilitate detecting a condition of a living body involve introducing a symbiotic agent into a living body; detecting a signal from the symbiotic agent indicative of the condition of the living body; and correlating the signal with the condition. Since various conditions have discrete signals, correlation is straight forward.

In accordance with another aspect of the claimed subject matter, methods that facilitate treating a condition of a living body involve introducing a symbiotic agent into a living body and releasing a treatment from the symbiotic agent to treat the condition of the living body.

The following description and the annexed drawings set forth in detail certain illustrative aspects of the claimed subject matter. These aspects are indicative, however, of but a few of the various ways in which the principles of the innovation may be employed and the claimed subject matter is intended to include all such aspects and their equivalents. Other advantages and novel features of the claimed subject matter will become apparent from the following detailed description of the innovation when considered in conjunction with the drawings.

### BRIEF DESCRIPTION OF THE DRAWINGS

FIG. 1 illustrates a block diagram of an exemplary system that facilitates sensing, detecting, or treatment of a condition of a living body using a symbiotic agent.

FIG. 2 illustrates a block diagram of an exemplary system that facilitates sensing, detecting, or treatment of a condition of a living body using a symbiotic agent, a controller and data store.

FIG. 3 illustrates a block diagram of an exemplary system that facilitates sensing, detecting, or treatment of a condition of a living body using a symbiotic agent.

FIG. 4 illustrates a block diagram of an exemplary system that facilitates sensing, detecting, or treatment of a condition of a living body using a symbiotic agent and an analysis component.

FIG. 5 illustrates a block diagram of an exemplary system that facilitates sensing, detecting, or treatment of a condition of a living body using a symbiotic agent and artificial intelligence.

FIG. 6 illustrates a block diagram of an exemplary system that facilitates sensing, detecting, or treatment of a condition of a living body using a symbiotic agent and alarms, logging, and/or querying.

### DETAILED DESCRIPTION

The claimed subject matter is described with reference to the drawings, wherein like reference numerals are used to refer to like elements throughout. In the following description, for purposes of explanation, numerous specific details are set forth in order to provide a thorough understanding of the subject innovation. It may be evident, however, that the claimed subject matter may be practiced without these specific details. In other instances, well-known structures and devices are shown in block diagram form in order to facilitate describing the subject innovation.

Compared to conventional gene therapy techniques, improved flexibility and increased functionality are achieved by genetically engineering symbiotic agents that can live naturally in a living body. The genetically engineered symbiotic agents can be removed when desired, such as when an intended purpose is complete or when an expected function is not achieved. The symbiotic agents sense, signal, provide and/or regulate health oriented substances or disease targeting substances. The subject innovation provides platforms for treating and detecting diseases deficiencies. In a specific embodiment, a human oriented disease fighting control system and/or human oriented metabolic control system that communicates with the body's biochemical systems and allows more flexible gene therapy.

A symbiotic agent is capable of existing in a living body for a desired period of time and capable of at least one of monitoring, sensing, signaling, and/or treating various conditions of the living body. Often times, a base agent which is genetically transformed into a symbiotic agent naturally exists in the living body, such as naturally occurring E. coli. Examples of symbiotic agents include genetically engineered or otherwise modified biological material. General examples of symbiotic agents include bacteria, viruses, fungi, algae, ciliates, parasites, pathogens, microorganisms, synthetic material such as hybridomas, and the like. A general example of bacteria specifically includes friendly bacteria. It is noted that symbiotic agents specifically include parasites, parasitic bacteria, and pathogenic bacteria since the positive effects and advantages outweigh the negative effects and disadvantages, especially when the negative effects and disadvantages of parasites, parasitic bacteria, or pathogenic bacteria can be reduced or minimized. In other words, although “parasitic” and “pathogenic” may not conventionally be interpreted as symbiotic, for purposes of this disclosure, symbiotic includes parasitic or pathogenic when there is a benefit associated with the parasitic symbiote or pathogenic symbiote.

Genetically engineered refers to any recombinant method used to create a host cell or organism (in this case the symbiotic agent) that expresses a protein or produces a compound or signal of interest. Methods and vectors for genetically engineering symbiotic agents are known. Examples of genetic engineering techniques include expression vectors, targeted homologous recombination and gene activation, and transactivation by engineered transcription factors.

Specific examples of symbiotic agents include bacteria of the genus Escherichia such as E. coli, bacteria of the genus Staphylococcus such as S. epidermis and S. aureus, those of the genus Arthrobacter such as A. agilis, A. citreus, A. globiformis, A. leuteus, A. simplex, those of the genus Azotobacter such as A. chroococcum, A. paspali, A. brasiliencise, and A. lipoferum, those of the genus Bacteroides such as B. lipolyticum and B. succinogenes, those of the genus Brevibacterium such as B. lipolyticum and B. stationis, those of the genus Kurtha such as K. zopfil, those of the genus Myrothecium such as M. verrucaria, those of the genus Pseudomonas such as P. calcis, P. dentrificans, P. flourescens, and P. glathei, those of the genus Phanerochaete such as P. chrysosporium, those of the genus Streptomyces such as S. fradiae, S. cellulosae, and S. griseoflavus, those of the genus Eubacterium, bacteria of the genus Bifidobacterium such as B. adolescentis, B. bifidum, B. animalis, B. thermophilum, B. breve, B. longum, B. infantis and B. lactis, Clostridium, Enterococcus, Fusobacterium, Peptostreptococcus, Ruminococcus, bacteria of the genus Lactobacillus such as L. acidophilus, L. bifidum, L. bulgaris, L. casei, L. bifidolongum, L. fermentum, L. brevis, L. cellobiosus, L. crispatus, L. curvatus, L. GG, L. gasseri, L. johnsonii, L. plantarum, L. salivarus, L. reuteri, L. delbrueckii, L. rhamnosusthose, those from the genus Saccharomyces (yeasts) such as S. cerevisiae, bacteria of the genus Actinomycetes, bacteria of the genus Azotobacter, bacteria of the genus Bacillus such as B. brevis, B. macerans, B. pumilus, B. polymyxa, B. subtilis, B. anthracis and B. cereus, bacteria of the species Bacteroides, bacteria of the genus Bordetella such as B. pertussis, bacteria of the genus Borrelia such as B. burgdorferi, bacteria of the genus Corynebacterium such as C. xerosis and C. hoffmanni, bacteria of the genus Campylobacter such as C. jejuni, those of the genus Candida such as C. albicans, Cyanobacteria, bacteria of the genus Deinococcus such as D. radiodurans, bacteria of the genus Enterococcus, bacteria of the genus Micrococcus, bacteria of the genus Streptococcus such as S. thermophilous and S. faecium, those of the genus Oceanobacillus, those of the genus Synechocystis, those of the genus Pseudomonas, those of the genus Helicobacter, those of the genus Mycobacterium, those of the genus Arabidopsis, those of the genus Thermotoga, and the like.

An additional source of cell lines and other biological material for use as symbiotic agents or as starting material to make symbiotic agents may be found in ATCC Cell Lines and Hybridomas, Yeast, Mycology and Botany, and Protists: Algae and Protozoa, and others available from American Type Culture Co. (Rockville, Md.), all of which are herein incorporated by reference.

Parasites include human parasites and non-human parasites such as animal parasites. Parasites are adept at penetrating into and residing in living body hosts. Owing to the natural affinity of parasites to specific areas of a living body, parasitic symbiotic agents can be advantageous in some situations where a specific organ or tissue of the living body is targeted for treatment, detection, or investigation. In instances where the treatment includes oncological treatments of a specific organ, using parasitic symbiotic agents is particularly advantageous. When using a parasitic symbiotic agent, it is in some instances helpful to mitigate the pathogenic affects of the parasite.

Specific examples of parasitic symbiotic agents include Balantinium coli (which typically burrows into the intestinal mucosa), trypanosoma brucei (a hemoflagellate), those of the genus Plasmodium (intracellular parasites of liver and red blood cells), nematodes such as Ascaris lumbricoides (roundworm which typically invades the gastrointestinal tract and lungs), pinworms such as Enterobius vermicularis (gastrointestinal tract, colon, fingertips), whipworms such as Trichuris trichiuria (gastrointestinal tract), flukes or trematodes such as Fasciola hepatica and schistosomes (liver and gallbladder), tapeworms or cestodes such as those from the genus Taenia (gastrointestinal tract), hookworms, heart worms, roundworms, Trichomonas vaginalis (vagina, urethra, epididymis, and prostate), spirochetes such as saprospira, cristispira, treponema (blood and lymphatic systems), lice (head, body, and pubic), scabies, those from the genus Entamoeba such as E. histolytica, those of the genus Cryptosporidium, those of the genus Schistosoma such as S. mansoni, S. haematobium, and S. japonicum, those of the genus Borrelia such as B. burgdorferi (brain), and the like.

The symbiotic agent can be such that indefinite presence in the living body is possible, or that temporary presence is possible (for example, if the symbiotic agent is ingested as a part of food, the symbiotic agent may pass through the digestive tract and exit the living body in the feces). An advantage associated with the symbiotic agent is that the duration or presence in the living body is controllable. In other words, techniques exist for removing the symbiotic agent from the living body when desired.

Symbiotic agents can be made using known techniques. For example, recombinant DNA techniques may be employed to make a genetically engineered bacterium having a desired function. Recombinant DNA is typically in the form of a vector. The vector may for example be a plasmid, cosmid, phage, or artificial chromosome. Vectors frequently include one or more selectable markers to enable selection of cells transformed (or transfected: the terms are used interchangeably in this specification) with them and, preferably, to enable selection of cells harboring vectors incorporating heterologous DNA. Appropriate “start” and “stop” signals are typically present. Additionally, if the vector is intended for expression, sufficient regulatory sequences to drive expression are present. Cloning vectors can be introduced into friendly bacteria, E. coli, or another suitable host which facilitates their manipulation.

Nucleic acid sequences for making the genetically engineered symbiotic agent may be prepared by any convenient method involving coupling together successive nucleotides, and/or ligating oligo- and/or poly-nucleotides, including cell-free in vitro processes, but recombinant DNA technology forms the method of choice. Ultimately, nucleic acid sequences can be introduced into host cells by any suitable means.

Nucleic acid sequences can be introduced into host cells by transformation using known techniques. Alternatively, nucleic acid sequences can be introduced into host cells by electroporation and vacuum filtration procedures. Alternatively, foreign DNA can be introduced directly into host cells using an electrical discharge apparatus. Any other suitable method that provides for the stable incorporation of the nucleic acid sequence within the nuclear DNA or mitochondrial DNA of host cells can be employed.

Examples of conditions or components that can be monitored, sensed, and/or treated include vitamin levels/production, mineral levels/production, amino acid levels/production/reduction, fatty acid levels/production, hormone levels/production, insulin levels/production, hemoglobin levels/production, glucose levels, alcohol levels, infections including viral infections and bacterial infections, parasitic infections, genetic disorders, cancer (colon cancer, liver cancer, bone cancer, brain cancer, stomach cancer, prostate cancer, breast cancer, ovarian cancer, cervical cancer, lung cancer, etc.), cancer produced substances such as various toxins, the undesirable presence of toxins, abnormal physical states, allergen levels (such as to insect bites/stings, pest/snake bites, food allergies, and the like), drug metabolite levels, immune response compounds (such as IgG, IgM, IgA, IgD, or IgE, etc.) levels/production, melanin levels/production, and the like.

The conditions are generally conditions of the living body, but also specifically include the conditions within local areas/tissues such as monitoring, sensing, signaling, and/or treating various conditions of the blood, plasma, digestive tract, feces, liver, lungs, lymphatic system, muscle tissue, bone marrow. Thus, generally speaking, detectable conditions include the presence, absence, or amount (including too high or too low) of a biologically necessary substance or a pathogen.

Treatment can include the release or use of a co-factor. A co-factor can be e.g., a chemical agent, a pharmaceutical, a protein, a peptide, that enables a biological system within a living body to perform or to perform in a more efficient manner. For example, a co-factor can increase the permeability of the capillaries in the intestinal mucosa to allow a sensing by bacteria of blood glucose levels, or changes in a selective manner the absorption of insulin from the gut.

The treatment of conditions of the living body by the symbiotic agent in a controllable manner is a significant advantage associated with the subject innovation. Treating in a controllable manner means that the actual treatment is provided in response to a stimulus or signal. For example, the symbiotic agent may release a compound for the treatment of a condition when a signal is received from an interface external to the living body. Alternatively, the symbiotic agent may release a compound for the treatment of a condition when a state is detected within the living body (e.g., a pH exceeding a threshold pH, a glucose level exceeding a threshold glucose level, the presence of toxin, etc.).

Monitoring and sensing conditions involves regulation or detection of materials that are indicative of a condition of the living body. The capability to sense a given condition can be activated or deactivated using a stimulus external to the living body. For example, a stimulus such as light, radiation, electromagnetic (EM) signals, or radio waves may be used to induce the symbiotic agent to sense for a given condition. For instance, an EM technique such as ultrasound can be implemented. In relation to EM and/or ultrasound an alignment can be changed. In general, the structure and/or activity of proteins or other molecules can be temporarily or permanently changed via changing a conformational structure, disrupting bonds at various energies (e.g., resonances, etc.), and so on. Furthermore, the subject innovation can be employed to turn off and/or on pathways or to leave permanent or semi-permanent bits around in response to a burst of EM and/or ultrasonic energy.

The capability of symbiotic agent to signal a condition permits communication of valuable information, often real time information, from within the living body. The capability of symbiotic agent to signal a condition permits communication of information that otherwise may not exist due to the absence of indicators readily detectable by casual human observation. Such information is valuable in that it is unbiased and free from subjective interpretation, thereby mitigating a human beings ability to misrepresent a condition of the body. Signaling is one or two way in that it alternatively or additionally includes the capability to signal the symbiotic agent external to living body. The capability of signaling the symbiotic agent permits the symbiotic agent to act, such as treat a condition, based on a stimulus from outside the living body (such as after diagnosis). The capability to signal a condition or signal the symbiotic agent may be controlled external to living body.

Examples of signals include chemical signals, radiation or light signals, electromagnetic signals, fluoresce, a detectable physical change in the living body (color change) and the like. A chemical signal can be generated by secretion of a detectable chemical, peptide, or protein (the detectable chemical or protein can be present in a blood sample, feces sample, etc.). The signal can not only communicate the presence of a condition (such as the detection of a low glucose level or the presence of a cancer produced substance), but the signal can also communicate specific details of a condition (such as the specific glucose level or the amount of a cancer produced substance) based on, for example, the intensity of the signal. Different chemical signals can be produced by the same symbiotic agent to indicate different conditions (that is, a given symbiotic agent may monitor, sense, signal, and/or treat more than one condition).

Living bodies are organisms that advantageously host a symbiotic agent. A prime example of a living body is a human being, although additional examples include other non-human mammals such as pets and livestock.

The symbiotic agents can be introduced into the living body by oral ingestion (such as in food), rectal, vaginal, injection, topical or transdermal, and the like. The mode of administration is not critical to the invention.

The symbiotic agents can be removed or deactivated by a suitable dosage of a disinfecting compound such as an antibiotic. The disinfecting compound either destroys, deactivates, or otherwise causes the symbiotic agent to leave the living body without deleteriously affecting the living body. Examples of the disinfecting compound include chemical biocides, disinfectants, antibiotics, and chemotherapeutic agents. Alternatively, the symbiotic agent can be engineered to self destruct or initiate apoptosis under controllable conditions, such as when a self destruct signal is received or the conditions in the living body activate a self destruct mechanism.

The living body may contain two or symbiotic agents having different functions. For example, a first colony of symbiotic agents may detect or sense glucose level and excrete an activation substance when the glucose level is too high. An activation substance is essentially a chemical trigger that induces another symbiotic agent or other cell to do something, such as make a substance. A second colony of symbiotic agents sensitive to the activation substance produced by the first colony of symbiotic agents may produce insulin when activated by the activation substance. Once the glucose level is within a normal threshold, the first colony of symbiotic agents ceases excretion of the activation substance and consequently the second colony of symbiotic agents ceases producing insulin. As a further example, a third colony of symbiotic agents, also sensitive to an activation substance or insulin, may facilitate delivery of the insulin produced by the second colony of symbiotic agents to the blood stream (for example, if the three colonies of symbiotic agents are located in the intestinal tract, the third colony may produce a substance that facilitates the migration/movement of insulin out of the intestinal tract and to the blood stream).

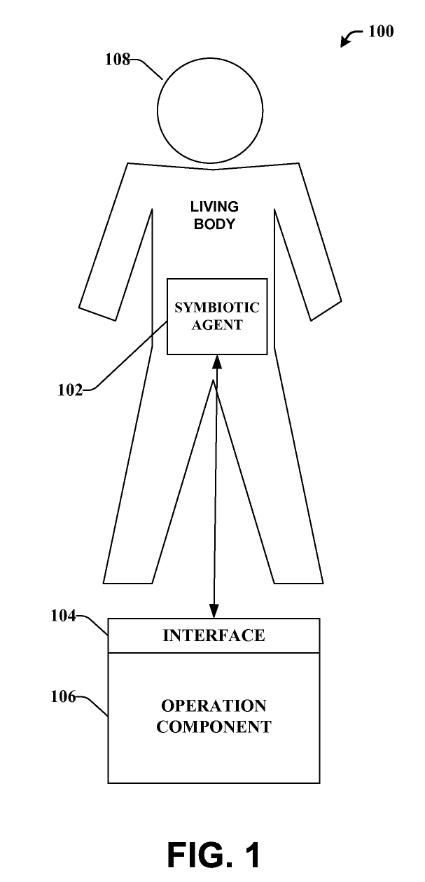

Turning now to the drawings, FIG. 1 illustrates a system 100 that facilitates implementing a symbiotic agent within a living body. A symbiotic agent 102 can provide a real-time in situ sensing and/or measurement related to one or more particular conditions, wherein the condition can be associated with the living body and/or external factors affecting the living body. The symbiotic agent 102 can be within or on the living body 108 such that various conditions can be sensed, measured, and/or detected, optionally in real-time, to provide accurate and unbiased data associated therewith. An operation component 106 (or artificial processing center) can analyze the data received or generated by the symbiotic agent 102 within the living body 108 via an interface 104 (discussed infra). The operation component 106 can provide a plurality of analysis based at least in part upon the received data related to a condition associated with the living body 108. In one example, the operation component can effectuate the living body 108 based at least in part upon the data collected or generated by the symbiotic agent 102. In other words, the operation component 106 can provide at least one of the following: data manipulation, updates, feedback, closed-loop control, state assessment (e.g. diseased), trending, forecasting, and/or any other suitable data configuration based on the symbiotic agent 102 and real-time detections. For example, the operation component 106 can manipulate any process and/or application associated with the living body 108, wherein the manipulation can be based at least in part upon the real-time in situ measurement of data collected by the symbiotic agent 102. The manipulation may be directed by one or more objectives such as required healthy living standards, standard human conditions, for example.

The symbiotic agent 102 can be placed within the living body to provide in situ measurements, wherein the operation component 106 can analyze such data to determine at least one condition of the living body. The symbiotic agent 102 can be placed, for instance, within the gastrointestinal tract, lungs, bladder, circulation system, muscle system, bone marrow, head, fatty tissue, or other internal organs. With the symbiotic agent 102 collecting data associated with the living body, the operation component 106 can provide diagnoses, recommendations, interventions, or other manipulations based at least in part upon the data generated by the symbiotic agent 102. It is to be appreciated that the symbiotic agent 102 within the living body can also signal a condition.

Furthermore, the system 100 can include any suitable and/or necessary interface component 104 (herein referred to as “interface 104”), which provides various adapters, collectors, connectors, channels, communication paths, receivers, etc. to integrate the operation component 106 into virtually any operating and/or database system(s). The interface 104 can receive data, wherein the data received can relate to the symbiotic agent 102; the living body 108; data associated with the living body; systems associated with the living body 108; collected data about a condition; any suitable data related to a living body condition, etc. In addition, the interface component 104 can provide various adapters, collectors, connectors, channels, communication paths, receivers, etc., that provide for interaction with the operation component 106. The results of the sensed information may be used to direct future analysis and/or symbiotic agent operation. For example, a change in chemistry detected by the symbiotic agent may result in a signal to further “probe” the living body for particular species representing the same or different condition that may be causing the change in chemistry.

The interface 104 may be used to send signals to the symbiotic agent 102. These signals enable the symbiotic agent 102 to function as an active sensor. For example, a non-human entity is detected in the vicinity of the symbiotic agent. This detection may be for example initiated by signals to the symbiotic agent 102. The characteristics of the signals may change over time and may be defined by operation component 106. The signal can cause the symbiotic agent to dynamically alter the chemistry thereby addressing the non-human entity.

It is to be appreciated that the system 100 can include a plurality of symbiotic agents 102 and the system 100 depicted is not to be limiting. For instance, the system 100 can include a plurality of symbiotic agents 102 with a single interface 104; multiple symbiotic agents 102 with multiple interfaces 104 and a plurality of operation components 106; etc. In other words, the system 100 can utilize any suitable number of symbiotic agents, interfaces, and/or sensor operation components to track, monitor, collect, and/or record, in situ data associated with the living body 108. Moreover, it is to be appreciated that the interface component 104 and the operation component 106 can be stand-alone components and not embedded in the living body environment.

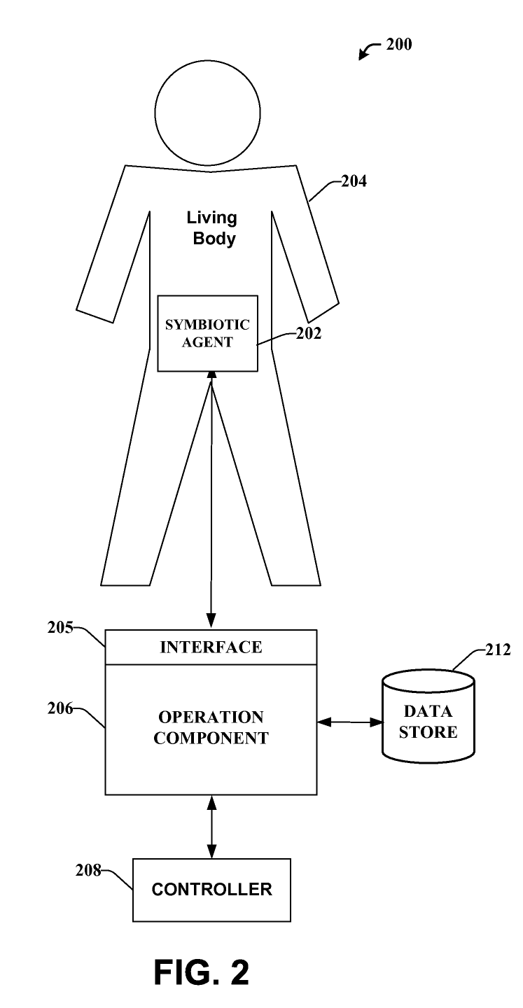

FIG. 2 illustrates a system 200 that facilitates detecting characteristics associated with a living body that utilizes a symbiotic agent. The system 200 includes a living body 204 that includes a symbiotic agent 202 that provides data associated thereto via an interface 205. The symbiotic agent 202 can measure various conditions, or sense various conditions, associated with the living body 204 to provide control, diagnosis, improved safety, improved health quality, and/or improved operation. Furthermore, a operation component 206 can provide analysis, processing, etc. based at least in part upon the data collected or generated by the symbiotic agent 202. It is to be appreciated that the symbiotic agent 202, the living body 204, and the operation component 206 can be substantially similar to the symbiotic agent 102, the living body 108, and the operation component 106 of FIG. 1, respectively. Although a single symbiotic agent 202 is illustrated in the system 200, it is to be appreciated and understood that the claimed subject matter is not so limited and any number of symbiotic agents can be utilized. The multiple symbiotic agents may operate by exchanging data and/or analysis between each other or the operation component.

The in situ data collected, detected, generated, signaled and/or monitored data related to the living body 204 can be analyzed and/or processed by the operation component 206. Furthermore, the operation component 206 can provide data manipulations based at least in part upon the data collected, generated, detected, and/or monitored by the symbiotic agent 202 within the living body 204. Moreover, the data collected, generated detected, and/or monitored can be utilized by a controller 208 coupled to the operation component 206. In accordance with an aspect of the subject innovation, the controller 208 can be a programmable logic controller (PLC). While PLCs can be utilized within the system 200 as the controller 208, it is to be understood that any suitable automation controller can be employed in connection with the claimed subject matter. For example, any suitable microprocessor and/or microcontroller can be utilized within the system 200 as the controller 208 including a personal computer or single-board computer with suitable input-output circuitry. Moreover, it is to be appreciated that the controller 208 can contain software components and hardware components having inputs and/or outputs that can be utilized in connection with analyzing or identifying conditions of the living body.

The operation component 206 can utilize a data store 212, wherein the data store 212 can store various data related to the system 200. The data store 212 can provide storage for in situ data collected via the symbiotic agent 202, configurations, conditions, health standards, control signal, system response, quality, disease/illness information, profiles, historic data, calibration data, security data, safety data, etc. The data store 212 can be, for example, either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable programmable ROM (EEPROM), or flash memory. Volatile memory can include random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), Rambus direct RAM (RDRAM), direct Rambus dynamic RAM (DRDRAM), and Rambus dynamic RAM (RDRAM). The data store 212 of the subject systems and methods is intended to comprise, without being limited to, these and any other suitable types of memory. In addition, it is to be appreciated that the data store 212 can be a server, a database, a hard drive, and the like.

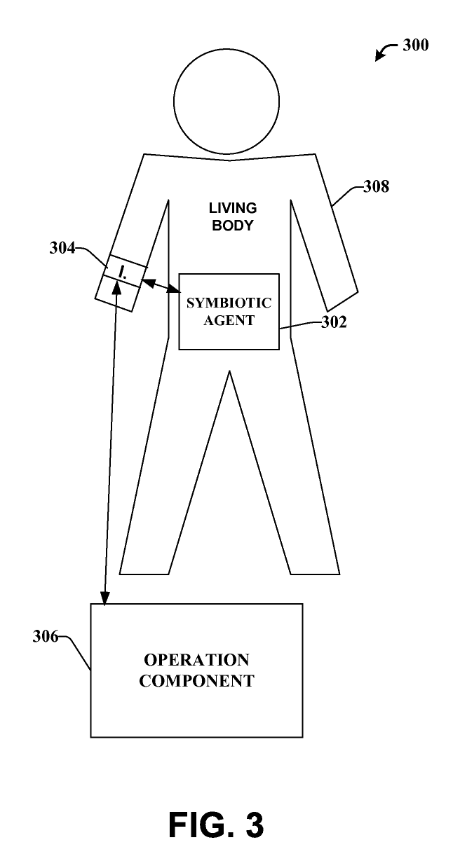

FIG. 3 illustrates a system 300 that facilitates implementing a symbiotic agent within a living body using an interface closely associated with a living body. A symbiotic agent 302 can provide sensing and/or measurement related to one or more particular conditions, wherein the condition can be associated with the living body and/or external factors affecting the living body. The symbiotic agent 302 can be within or on the living body 308 such that various conditions can be sensed, measured, and/or detected, optionally in real-time, to provide accurate and unbiased data associated therewith. An operation component 306 (or artificial processing center) can analyze the data received or generated by the symbiotic agent 302 within the living body 308 via an interface 304 (discussed infra).

The system 300 can include any suitable and/or necessary interface component 304 (herein referred to as “interface 304”), which provides various adapters, collectors, connectors, channels, communication paths, receivers, transmitters, etc. to integrate the operation component 306 with the symbiotic agent and into virtually any operating and/or database system(s). The interface 304 can receive data, wherein the data received can relate to the symbiotic agent 302; the living body 308; data associated with the living body; systems associated with the living body 308; collected data about a condition of the living body; any suitable data related to a living body condition, etc. In addition, the interface component 304 can provide various adapters, collectors, connectors, channels, communication paths, receivers, transmitters, etc., that provide for interaction with the operation component 306. While the interface 304 as shown is an arm band or watch, the interface may be positioned at any location of the living body most suitable to communicate with the symbiotic agent 302. For example, the interface 304 may be incorporated into a hat, glasses, a ring, shoes, an ankle strap, belt, other clothing articles, The interface 304 may be positioned within the living body 308, such as within a pacemaker, a cochlear implant, artificial joint/limb, etc.

The interface 304 may be used to send signals to the symbiotic agent 302. These signals enable the symbiotic agent 302 to function as an active sensor. For example, a non-human entity such as a pathogen, may be detected in the vicinity of the symbiotic agent. This detection may be for example initiated by signals to the symbiotic agent 302 from the interface 304. The signal can cause the symbiotic agent to dynamically alter the chemistry in the living body thereby addressing the non-human entity.

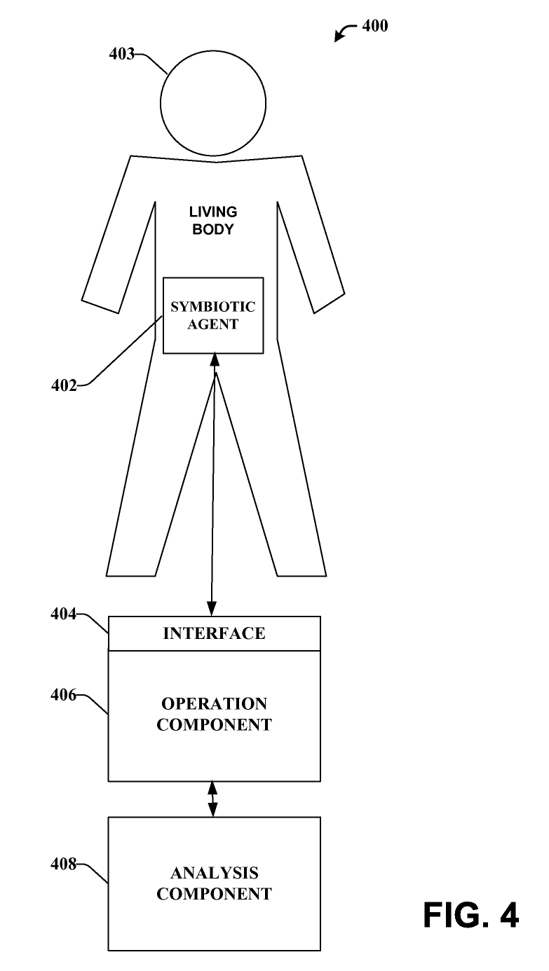

FIG. 4 illustrates a system 400 that facilitates detecting illness conditions within a living body. The system 400 can include an in situ symbiotic agent 402 that provides continuous sensing, monitoring and/or detection in a living body 403 includes a symbiotic agent 402. It is to be appreciated that the symbiotic agent 402, operation component 406, and the analysis component 408 can be substantially similar to previously described components. The analysis component 408 can analyze data generated by the symbiotic agent 402 in order to identify an illness or disease. For example, the symbiotic agent 402 can include a number of different receptors specific for various different maladies. The symbiotic agent 402 signals the operation component 406 via the interface 404 with data that can be extrapolated to determine which receptor is occupied, and what the significance of the bound receptor is. Alternatively, the symbiotic agent 402 produces a signal compound indicating to the operation component 406 via the interface 404 that a certain receptor is experiencing a binding event. Although the interface 404 is shown directly coupled with the operation component 406, the interface 404 may be directly coupled with the living body 403, such as a device held within a wrist band, ankle band, clothing article, shoe, etc.

The analysis component 408 may contain a classifier. A classifier is a function that maps an input attribute vector, x=(x1, x2, x3, x4, xn), to a confidence that the input belongs to a class, that is, f(x)=confidence(class). Such classification can employ a probabilistic and/or statistical-based analysis and can further be influenced by a consideration of utilities or costs associated with correct and incorrect classification to prognose or infer an action that a user desires to be automatically performed. A support vector machine (SVM) is an example of a classifier that can be employed. The SVM operates by finding a hypersurface in the space of possible inputs, where the hypersurface splits the triggering criteria from the non-triggering events. Intuitively, this makes the classification correct for testing data that is near, but not identical to training data. Other model classification approaches include, e.g., naïve Bayes, Bayesian networks, decision trees, neural networks, fuzzy logic models, and probabilistic classification models assuming different patterns of independence can be employed. Classification as used herein also is inclusive of statistical regression that is utilized to develop models of priority.

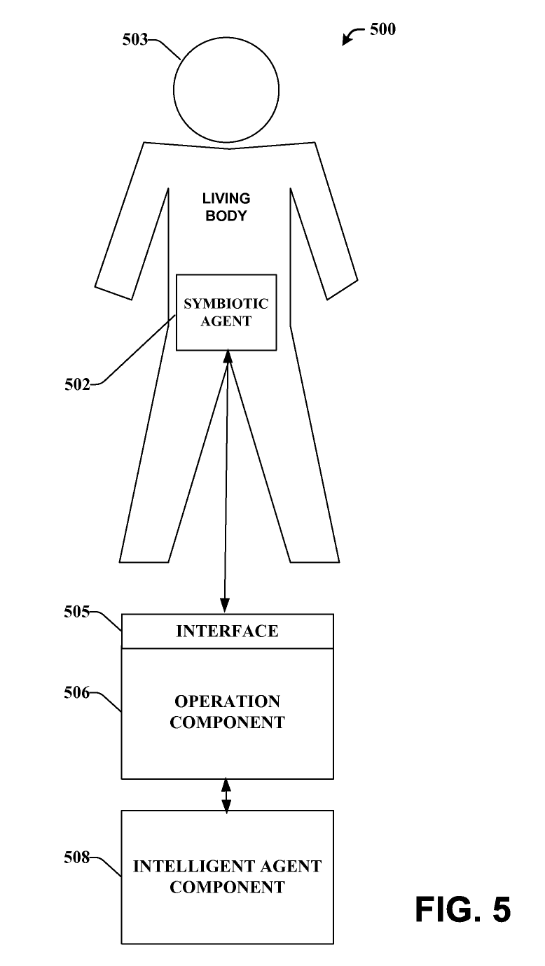

FIG. 5 illustrates a system 500 that facilitates monitoring a living body with symbiotic agent in real-time. The system 500 can include a symbiotic agent 502 that can be incorporated into a living body 503. An operation component 506 can provide data manipulations based at least in part upon the collected real-time data, wherein such manipulations can provide treatment, diagnosis, prognosis, control, detection, of a condition of the living body 503.

The system 500 can include an intelligent agent component 508 that can be deployed on commercially available PLC's, PC's, SBC, etc. within or connected to the operation component 506. It is to be appreciated that the claimed subject matter can include multiple symbiotic agents 502 and/or multiple intelligent agent components 508. Moreover, it is to be understood that the intelligent agent component 508 can be a stand-alone component or incorporated into the operation component 506. The intelligent agent component 508 can provide holonic system capabilities and the eventual transition to autonomous agents, which can respond to unexpected detections/data, the ability to dynamically respond to the unexpected detections/data. The intelligent agent component 508 can allow highly distributed diagnostics to sense an unfavorable condition and prescribe a superior countermeasure.

The intelligent agent component 508 can utilize a suite of simulation, prototyping, and/or deployment tools in accordance with the subject innovation. By deploying the intelligent agent component 508, unprecedented intervention can be provided as well as consequent overall health benefits. The system 500 can significantly enhance the quality, health, and efficacy of living bodies. Furthermore, the real-time process information can be utilized for closed-loop feedback control, adaptive process model development, predictive treatment, and other intervention applications.

The system 500 can enable the living body to function during illness, deficiency, or disease. Moreover, the intelligent agent component 508 can adhere to published agent-to-agent communication protocols (e.g., Foundation for Intelligent Physical Agents (FIPA)) and provide local intervention and decision-making along with more overall health goals.

It is to be understood that the intelligent agent component 508 can provide for reasoning about or infer states of the system, environment, and/or user from a set of observations as captured via events and/or data. Inference can be employed to identify a specific context or action, or can generate a probability distribution over states, for example. The inference can be probabilistic—that is, the computation of a probability distribution over states of interest based on a consideration of data and events. Inference can also refer to techniques employed for composing higher-level events from a set of events and/or data. Such inference results in the construction of new events or actions from a set of observed events and/or stored event data, whether or not the events are correlated in close temporal proximity, and whether the events and data come from one or several event and data sources. Various classification (explicitly and/or implicitly trained) schemes and/or systems (e.g., support vector machines, neural networks, expert systems, Bayesian belief networks, fuzzy logic, data fusion engines . . . ) can be employed in connection with performing automatic and/or inferred action in connection with the claimed subject matter.

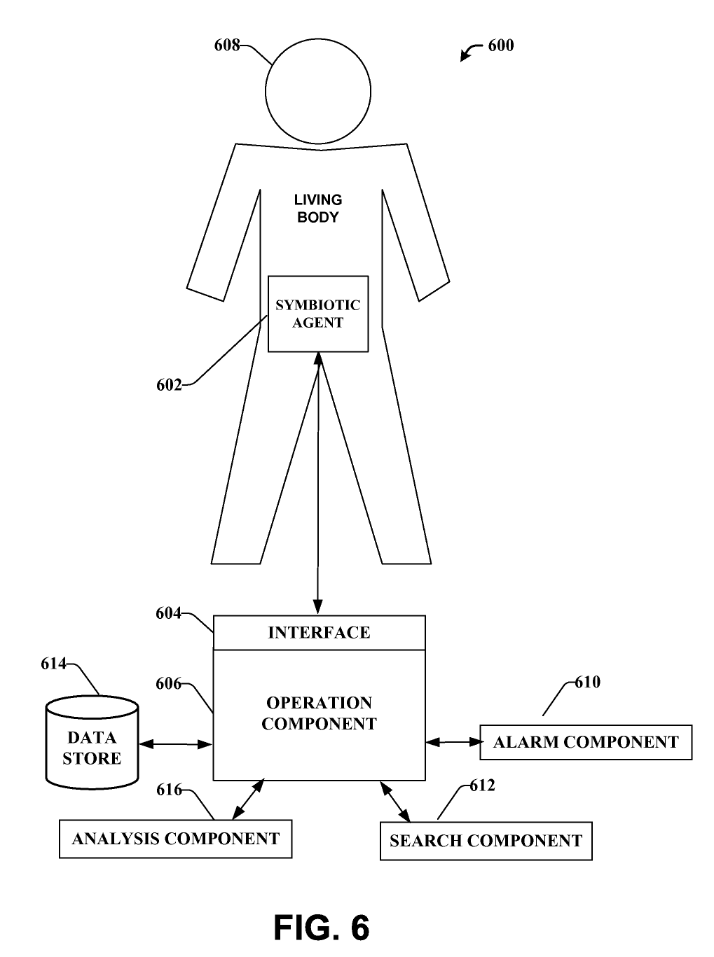

FIG. 6 illustrates a system 600 that facilitates implementing symbiotic agents within a living body 608 providing alarms, data logging/searching, event logging, and/or querying. The system 600 can include a symbiotic agent 602 that provides monitoring, detection, and/or data collection within a living body 608. The collected data can be utilized by an operation component 606, wherein the data can be implemented for analysis, computations, determinations, and/or any other manipulations based upon the collected data.

The operation component 606 can utilize an alarm component 610 that can provide alarms and/or warnings associated with data generated by the symbiotic agent based at least in part upon the health severity of the measurements, detections, and/or data collections via the symbiotic agent 602 and/or historical/nominal data. For example, the alarm component 610 can implement an alarm or unambiguous warning such as, warning lights, pop-up screens, blinking data display items, graphical items, email, text, cellular communication, web site activity, etc. when a particular condition is detected or measured beyond a specific threshold. It is to be appreciated that the status and/or alarms can be stored in the data store 614 (described previously), a “black box” recorder, etc. Additionally, the alarm component can signal a condition that may occur in the future to permit action to be taken before a condition occurs or before actions causing a condition are performed.

The operation component 606 can further utilize a search component 612 that allows querying of the system 600. In particular, the search component 612 can provide querying of any data collected or generated by the symbiotic agent 602, stored data, system data, conditions of the living body 608, analytical data, historical data and/or any other data related to the system 600. For instance, the search component 612 can be utilized to discover data related to a particular condition and circumstances present when the condition was measured/detected. It is to be appreciated that although the search component 612 is illustrated as a stand-alone component, the search component 612 can be incorporated into the operation component 606. It is to be appreciated that the system components can be distributed and remote from the living body 608 and remote from each other such as accessible via Internet, Ethernet, etc.

In addition, the search component 612 can further employ a log component wherein the log component can store and/or track various data related to the system 600. The system 600 can include an analysis component 616, which can analyze the significance of data generated by the symbiotic agent 602, and prescribed suitable countermeasures to conditions detected within the living body 608.

Data generated by symbiotic agents may be collected and analyzed, providing powerful information to devise new and alternative treatments and interventions for diseases, deficiencies, preventions, and so forth.

In one embodiment, the symbiotic agent is a friendly bacteria transformed with a gene that expresses insulin or proinsulin. However, the bacteria are genetically engineered so that expression of insulin or proinsulin only occurs when the blood glucose level exceeds a predetermined threshold amount, such as more than about 6.1 mmol/l. The genetically engineered symbiotic agent contains a glucose receptor for sensing the presence/amount of glucose. The symbiotic agent is administered to a human subject afflicted with diabetes (type 1 or type 2). Once in the human subject, the symbiotic agent senses its environment, constantly determining whether or not the blood glucose level exceeds or does not meet the predetermined threshold amount. When the blood glucose level does not meet the predetermined threshold amount, the symbiotic agent expresses nothing. When the blood glucose level exceeds the predetermined threshold amount, the symbiotic agent expresses insulin or proinsulin until the blood glucose level does not meet the predetermined threshold amount.

In a related embodiment, a first symbiotic agent is a friendly bacteria transformed with a gene that causes expression of a signal compound when blood glucose level exceeds a predetermined threshold amount and a second symbiotic agent is a friendly bacteria transformed with a gene that expresses insulin or proinsulin when a signal compound is detected. The second genetically engineered symbiotic agent contains a receptor for the signal compound, and when the signal compound binds to the receptor, expression of insulin or proinsulin is activated. The symbiotic agents are administered to a human subject afflicted with diabetes. Once in the human subject, the first symbiotic agent senses its environment, constantly determining whether or not the blood glucose level exceeds or does not exceed the predetermined threshold amount. When the blood glucose level does not meet the predetermined threshold amount, the first symbiotic agent expresses nothing. When the blood glucose level exceeds the predetermined threshold amount, the first symbiotic agent expresses a signal compound. The presence of the signal compound induces the second symbiotic agent to express insulin or proinsulin until the signal compound cannot be detected.

In another embodiment, the symbiotic agent is a friendly bacteria transformed with a gene that expresses a peptide that induces other bacteria to produce vitamin B12. The bacteria are genetically engineered so that expression of the peptide only occurs when vitamin B12 is not detected, or when the vitamin B12 level is less than a threshold amount, such as less than about 0.1 μg/l. The symbiotic agent is administered to a human subject afflicted with a vitamin B12 deficiency. Once in a human subject with a potential vitamin B12 deficiency, the symbiotic agent senses its environment, constantly determining whether or not vitamin B12 exists. When vitamin B12 is detected or sensed, the symbiotic agent expresses nothing. When no vitamin B12 is sensed, the symbiotic agent expresses a peptide that induces other bacteria to produce vitamin B12 until the vitamin B12 level becomes detectable.

In yet another embodiment, the symbiotic agent is L. acidophilus transformed with a gene that expresses a signal compound when blood is detected. L. acidophilus is selected for transformation due to its natural presence in the colon. The transformed L. acidophilus symbiotic agent contains a hemoglobin receptor for detecting the presence of red blood cells. The transformed L. acidophilus symbiotic agent is genetically engineered so that the binding of hemoglobin to the hemoglobin receptor activates expression of the signal compound. Once in a human subject with polyps in the colon, the symbiotic agent senses its environment, and produces a signal compound when blood is detected. The signal compound is readily detectable in feces excreted from the human subject. By monitoring for presence of the signal compound in feces, for example using a hand held detector, one is in fact monitoring for a symptom of colon cancer; namely, rectal bleeding. Since low levels of rectal bleeding are often difficult to detect, especially for those with untrained eyes, the symbiotic agent provides an alternative opportunity to detect colon cancer.

In related embodiment, the L. acidophilus can be transformed with several genes, each expressing a different signal compound when different conditions are sensed (such as the presence of blood, the presence of specific toxins, a deleterious increase in pH, etc.). By monitoring for presence of the signal compounds in feces one can monitor for a symptom of colon cancer (bleeding) as well as a dangerous increase in illness causing bacteria.

In still yet another embodiment, the symbiotic agent is a friendly bacteria transformed with a gene that expresses a signal peptide when even just a very small amount of pregnancy hormone human chorionic gonadotrophin (HCG) is present or sensed. The symbiotic agent contains an HCG receptor for detecting the presence of HCG. The symbiotic agent is genetically engineered so that the binding of HCG to the HCG receptor activates expression of the signal peptide. Once in a human subject that may be pregnant, the symbiotic agent senses for HCG, and produces a signal peptide when HCG is sensed. Since it is difficult to detect HCG using over-the-counter pregnancy tests in the early days of pregnancy, detecting the expressed peptide provides an early indication of pregnancy.

In still yet another embodiment, the symbiotic agent is a friendly bacteria transformed with a gene that expresses one or more alcohol dehydrogenases when ethyl alcohol is present. The symbiotic agent has a receptor that binds specifically with alcohol. When the alcohol binds to the alcohol receptor, alcohol dehydrogenase expression is activated. Once in a human subject, the symbiotic agent senses its environment, and produces one or more alcohol dehydrogenases when alcohol is sensed. The symbiotic agent can then lower the toxic effect of alcohol consumed by the human subject.

In still yet another embodiment, the symbiotic agent is a liver fluke genetically engineered to secrete a chemotherapeutic compound when alpha-fetoprotein is present or sensed. The symbiotic agent contains an alpha-fetoprotein receptor for detecting the presence of alpha-fetoprotein (a generally accepted marker of a liver tumor). The symbiotic agent is genetically engineered so that the binding of alpha-fetoprotein to the alpha-fetoprotein receptor activates secretion of the chemotherapeutic compound, which is effective in treating liver cancer. Once in a human subject, the symbiotic agent naturally migrates to the liver, senses for alpha-fetoprotein, and produces a chemotherapeutic compound when alpha-fetoprotein is sensed.

In another embodiment, the symbiotic agent is Ascaris lumbricoides genetically engineered to produce oxygen (O₂) when activated by an electromagnetic wave of predetermined frequency. Once in a human subject afflicted with a severe case of pneumonia, the symbiotic agent naturally migrates to the lungs. Under application of the electromagnetic signal of predetermined frequency, the Ascaris lumbricoides symbiotic agent produces oxygen which facilitates respiration.

In yet another embodiment, the symbiotic agent is cristispira genetically engineered to secrete a compound that moderates multiplication of lymphocytes when in an electromagnetic field of sufficient flux. Lymphomas result when cells in the immune system multiply uncontrollably. Once in a human subject with lymphoma, the symbiotic agent naturally migrates to the lymphatic system. Under application of an electromagnetic field from outside the human subject, the cristispira symbiotic agent secretes the compound that moderates multiplication of lymphocytes.

It is to be appreciated and understood that directions in synthetic biology are enabling increasingly flexible and reusable specifications of systems based on inventories of well-characterizable components and assemblies such as channels, timers, switches, etc. Moreover, introduced biological systems can employ components that record and/or remember multiple states over time, as encoded in a single and/or multiple molecules.

The techniques, systems, and methods described herein further include engineering colonies or, more generally, systems of different types of bacteria and/or parasites. The properties of such larger systems provide designers more flexibility to sense, produce, or intervene. In general, the rich control and generative machinery of symbiotes—as well as, where necessary pathogens and parasites are exploited to perform the desired tasks that improve the overall health of living bodies.

As utilized herein, terms “component,” “system,” “interface,” “device,” “generator,” “collector,” and the like are intended to refer to a computer-related entity, either hardware, software (e.g., in execution), and/or firmware. For example, a component can be a process running on a processor, a processor, an object, an executable, a program, a function, a library, a subroutine, and/or a computer or a combination of software and hardware. By way of illustration, both an application running on a server and the server can be a component. One or more components can reside within a process and a component can be localized on one computer and/or distributed between two or more computers.

Furthermore, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, carrier, or media. For example, computer readable media can include but are not limited to magnetic storage devices (e.g., hard disk, floppy disk, magnetic strips . . . ), optical disks (e.g., compact disk (CD), digital versatile disk (DVD) . . . ), smart cards, and flash memory devices (e.g., card, stick, key drive . . . ). Additionally it should be appreciated that a carrier wave can be employed to carry computer-readable electronic data such as those used in transmitting and receiving electronic mail or in accessing a network such as the Internet or a local area network (LAN). Of course, those skilled in the art will recognize many modifications may be made to this configuration without departing from the scope or spirit of the claimed subject matter. Moreover, the word “exemplary” is used herein to mean serving as an example, instance, or illustration. Any aspect or design described herein as “exemplary” is not necessarily to be construed as preferred or advantageous over other aspects or designs.

While the claimed subject matter has been described above in the general context of computer-executable instructions of a computer program that runs on a local computer and/or remote computer, those skilled in the art will recognize that the subject innovation also may be implemented in combination with other program modules. Generally, program modules include routines, programs, components, data structures, etc., that perform particular tasks and/or implement particular abstract data types.

Moreover, those skilled in the art will appreciate that the inventive methods may be practiced with other computer system configurations, including single-processor or multi-processor computer systems, minicomputers, mainframe computers, as well as personal computers, hand-held computing devices, microprocessor-based and/or programmable consumer electronics, and the like, each of which may operatively communicate with one or more associated devices. The illustrated aspects of the claimed subject matter may also be practiced in distributed computing environments where certain tasks are performed by remote processing devices that are linked through a communications network. However, some, if not all, aspects of the subject innovation may be practiced on stand-alone computers. In a distributed computing environment, program modules may be located in local and/or remote memory storage devices.

What has been described above includes examples of the subject innovation. It is, of course, not possible to describe every conceivable combination of components or methodologies for purposes of describing the claimed subject matter, but one of ordinary skill in the art may recognize that many further combinations and permutations of the subject innovation are possible. Accordingly, the claimed subject matter is intended to embrace all such alterations, modifications, and variations that fall within the spirit and scope of the appended claims.

In particular and in regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the innovation includes a system as well as a computer-readable medium having computer-executable instructions for performing the acts and/or events of the various methods of the claimed subject matter.

There are multiple ways of implementing the present innovation, e.g., an appropriate API, tool kit, driver code, operating system, control, standalone or downloadable software object, etc. which enables applications and services to use the advertising techniques of the invention. The claimed subject matter contemplates the use from the standpoint of an API (or other software object), as well as from a software or hardware object that operates according to the advertising techniques in accordance with the invention. Thus, various implementations of the innovation described herein may have aspects that are wholly in hardware, partly in hardware and partly in software, as well as in software.

The aforementioned systems have been described with respect to interaction between several components. It can be appreciated that such systems and components can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described herein may also interact with one or more other components not specifically described herein but generally known by those of skill in the art.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

What is claimed is:

1. A system that facilitates sensing, detecting, or treatment of a condition of a living body, comprising:
a symbiotic agent (102);
an interface component (104) that facilitates receipt of or delivery of data associated with the condition of the living body (108); and
an operation component (106) that analyzes received data associated with the condition of the living body or provides data for delivery to the symbiotic agent.

2. The system of claim 1, the symbiotic agent comprising a genetically engineered bacterium.

3. The system of claim 1, the symbiotic agent comprising a genetically engineered parasite.

4. The system of claim 1, the living body comprising a human being.

5. The system of claim 1, the living body comprising a non-human mammal.

6. The system of claim 1, providing data for delivery to the symbiotic agent comprising a signal inducing the symbiotic agent to express a treatment.

7. The system of claim 1, receiving data associated with the condition of the living body comprising data indicative of a receptor binding event on the symbiotic agent.

8. A method that facilitates detecting a condition of a living body, comprising:
introducing a symbiotic agent into a living body;
detecting a signal from the symbiotic agent indicative of the condition of the living body; and
correlating the signal with the condition.

9. The method of claim 8, the signal comprising a compound produced by the symbiotic agent.

10. The method of claim 8, detecting the signal comprising contacting a product produced by the living body to chemical analysis.

11. The method of claim 8, the symbiotic agent comprising a receptor for a compound indicative of the condition of the living body.

12. A method that facilitates controllably treating a condition of a living body, comprising:
introducing a symbiotic agent into a living body; and
releasing a treatment from the symbiotic agent in a controlled manner to treat the condition of the living body.

13. The method of claim 12, releasing the treatment comprising the symbiotic agent producing a compound after activation by a light signal.

14. The method of claim 12, releasing the treatment comprising the symbiotic agent producing a compound in response to a receptor binding event on the symbiotic agent.

15. The method of claim 12, releasing the treatment comprising the symbiotic agent producing a compound after activation by an electromagnetic signal.
